(19) United States
(12) Patent Application Publication
(10) Pub. No.: US 2012/0078285 A1
(43) Pub. Date: Mar. 29, 2012

(54) BALLOON CATHETER FOR INTRAVASCULAR THERAPIES

(75) Inventor: Stephen Griffin, San Jose, CA (US)

(73) Assignee: Penumbra, Inc., Alameda, CA (US)

(21) Appl. No.: 13/076,707

(22) Filed: Mar. 31, 2011

Related U.S. Application Data
(60) Provisional application No. 61/319,926, filed on Apr. 1, 2010.

(51) Int. Cl. A61F 2/01; B23P 11/00

(52) U.S. Cl. 606/200; 29/428

(57) ABSTRACT

A low profile, tailored stiffness intravascular balloon catheter is disclosed for use especially in treatment of intracranial aneurysm. Treatment utilizing the device can be performed without the need for a guide wire during delivery of embolic implants. A profiled metal hyptotube that is machine-cut or laser cut in a dual, off-set helical pattern, is the foundation for the device. A polymer jacket may be disposed upon the hypotube. A thin wall elastomeric balloon is bonded to the distal end of the system in fluid communication with the hypotube. The system may have one or more delivery ports for the release of embolic implants.

### CROSS-REFERENCE TO RELATED APPLICATIONS

This application claims the benefit of provisional application No. 61/319,926, filed on Apr. 1, 2010, the full disclosure of which is incorporated herein by reference.

### BACKGROUND OF THE INVENTION

1. Field of the Invention

The present invention relates generally to medical devices and specifically to intravascular balloon catheters for treatment of vascular disease such as aneurysm, including for delivery of embolic implants.

Stroke is the third leading cause of death in the United States, and is the leading cause of disability. Types of stroke are divided into ischemic stroke and hemorrhagic stroke. Hemorrhagic stroke results from the rupture of an aneurysm in the wall of an intracranial vessel. Numerous intravascular procedures have been developed to treat both ischemic and hemorrhagic stroke. Most procedures employ the sequential introduction of a guide wire, a guide catheter, and subsequent therapy. In the treatment of hemorrhagic stroke, a balloon catheter is often used to accompany the delivery of embolic coils used to fill an aneurysm to prevent further blood flow into the aneurysm.

Current balloon catheter systems employ the guide wire for delivery and deployment of the inflatable balloon catheter and as a safety measure. Firstly, a guide wire is used for tracking the catheter to the treatment site. The guide wire is introduced first, and is used to “find” the path through the tortuous anatomy to the treatment site. The catheter is then tracked over the guide wire, which may provide a “rail” for or otherwise guide the catheter. Following tracking to a treatment site, the guide wire is then typically used during deployment of the balloon catheter. After the balloon is inflated within the vessel, the guide wire is used to confirm a tolerance fit for the balloon. The tolerance fit confirms an almost fluid seal for injection of contrast die. Further, the wire provides a fail-safe to permit balloon deflation in the event aspiration through the system lumen cannot be achieved. And finally, the interference fit of the wire may confirm that any residual air has been eliminated from the system. Current conventional systems to perform the procedure are typically 2.5 French in size.

In the aneurysm coil embolization procedure, a guide wire and catheter are introduced into the femoral artery and navigated through the vascular system to the site of the aneurysm under fluoroscopic visualization. In some instances, an inflatable balloon is used to secure the position of the catheter at the site of the aneurysm. Also, in some instances, a stent is deployed across the “neck” of the aneurysm, and coils are delivered from the catheter, through the interstices of the stent, and into the aneurysm.

However, in smaller diameter vessels, it is often difficult to “bridge” the neck of the aneurysm with a stent. Under these circumstances, it may be desirable to bridge the neck of the aneurysm with the balloon positioned at the distal end of the catheter. The inflatable balloon may be used to “remodel” the neck of the aneurysm and to secure the delivery catheter at the treatment site during delivery and deployment of the coils. The inflated balloon may further prevent escape of embolic implants during release into the aneurysm. Multiple coils may be introduced into a single aneurysm cavity for optimal filling of the cavity. The deployed coils serve to block blood flow into the aneurysm and reinforce the aneurysm against rupture.

2. Description of the Background Art

Catheters having tubular bodies with helical or other cuts to control flexibility are described in U.S. Pat. Nos. 7,785,289; 7,815,600; 6,074,407; 6,527,790; 6,293,960; and U.S. Patent Publication Nos. 2006/0084939; 2009/0157048; and 2004/0019322.

### BRIEF SUMMARY OF THE INVENTION

Catheters according to the invention herein may be used to remodel the neck of the aneurysm without requiring the use of a guide wire for deployment of a balloon or release of embolic implants. A catheter according to the invention will have a low profile allowing delivery using a guide catheter as small as 1.5 French. A catheter according to the invention will provide a balloon having a tailored stiffness that will permit it to serve some of the purposes of a guide wire. The interior volume of the balloon catheter disclosed herein is significantly smaller than currently marketed systems, rendering any residual air bubble negligible, and thereby further reducing the need for a guide wire for safety. Still further, a tailored balloon rupture pressure and/or a slow distal leak through the balloon distal tip will reduce the need for a guide wire for safety.

The inflatable balloon catheter of the present invention may be delivered through any appropriately sized guide catheter or microcatheter. The distal end of the microcatheter is introduced into the femoral artery through a small incision near the groin. Deployment of the disclosed balloon catheter system will be achieved by filling the balloon and catheter lumen using the same technique as conventional filling or inflating of PTCA balloon catheters. A distal end of the catheter may be positioned at the target site via the microcatheter, and the distal balloon inflated at the target site. Contrast dye may be injected into the catheter for enhanced visualization of the artery.

The novel balloon catheter design employs a profiled machine cut or laser cut metal hypotube which transitions to a softer profile towards the distal end by controlling the pitch of the tube. The cut pattern utilizes a dual helical or spiral cut where the second cut is offset from the first (typically being out-of-phase by an angle in the range from 90° to 180°) in order to provide an interference locking mechanism to provide stretch resistance in the tube while at the same time preserving lateral flexibility. A polymer jacket will be placed over at least a portion of the machine/laser cut hypotube to provide a fluid tight seal to allow delivery contrast to the balloon and to provide a bonding substrate to bond the proximal end of the balloon to the metal hypotube shaft. The polymer jacket will also provide a tie layer for the hydrophilic coating to adhere in order to enhance deliverability. The system may further be manufactured to have enhanced visibility.

### BRIEF DESCRIPTION OF THE DRAWINGS

FIG. 1 is a side view of an embodiment according to the invention in its deployed configuration.

FIG. 2 is a “see-through” side view of a component of the embodiment of FIG. 1.

FIG. 3 illustrates a balloon catheter system according to the invention within a vessel of a subject.

### DETAILED DESCRIPTION OF THE INVENTION

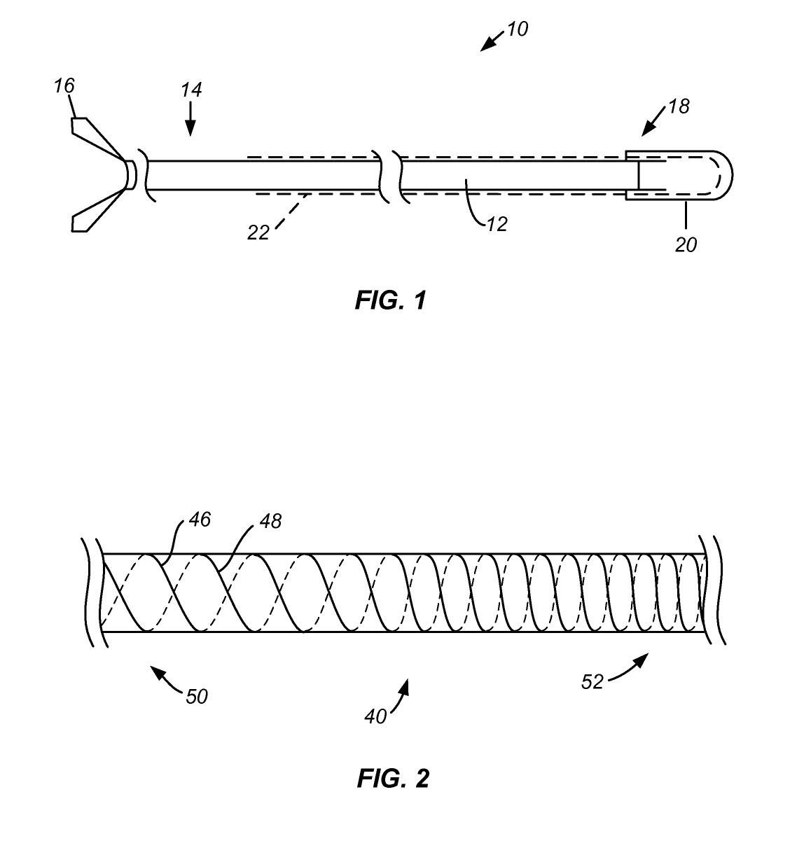

FIG. 1 illustrates a balloon catheter system according to the invention. Balloon catheter system 10 includes an elongate tubular member 12 extending from proximal end 14 to distal end 18, in fluid communication with inflatable balloon 20. Though not drawn to scale, a truncated version of system 10 is illustrated in FIG. 1 with balloon 20 in its inflated configuration. A balloon catheter according to the invention may be characterized as having any length, outer diameter and other dimensions that are suitable for carrying out procedures within the intracranial vasculature. In the example of FIG. 1, balloon catheter system 10 has a length ranging from 70 cm to 110 cm.

Proximal end 14 of tubular member 12 is attached to a standard hub or luer 16 or other comparable device for connection to a suitable means for delivery and withdrawal of inflation media. Tubular member 12 is provided with an inflation lumen (not shown) in fluid communication with balloon 20. A delivery port, (not pictured), for release of embolic coils or other embolic material may be disposed near the distal end of tubular member 12 and balloon 22. Alternatively, a delivery port may be disposed along the length of balloon 20, which may be lobed (and include openings between adjacent lobes) to permit unobstructed release of embolic material.

An exemplary balloon 20 is a sealed, compliant balloon constructed of a thin walled elastomer, typically a polyurethane thermoelastic elastomer such as ChronoThane™, available from AdvanSource Biomaterials of Wilmington, Mass., or other suitable material. The material can either be cast or extruded to a desired thickness in order to achieve a desired compliance and accommodate the expected inflation pressure. Typical balloon wall thicknesses will range from 0.00075 inch to 0.0015 inch, depending on the properties desired. Providing catheter system 10 with an atraumatic distal tip and tailored stiffness, balloon 20 provides some of the function of a guidewire in placement of the distal end 18 of balloon catheter system 10 in the distal vasculature and at the site of an aneurysm or other defect (not pictured in FIG. 1). The rupture pressure of balloon 20 may be selected to provide a safety feature, lessening the need for a guide wire to confirm a tolerance fit in the vessel. Balloon 20 may also be fabricated to allow a slow distal leak of inflation medium as an added safety feature to ensure reliable deflation of balloon 20 without the use of a guide wire, and to ensure safe removal of system 10 following the conclusion of a procedure.

The elastomer forming balloon 20 may be loaded with radiopaque material to enhance visibility under fluoroscopy. Alternatively, or in addition, one or more radiopaque marker bands may be disposed about balloon catheter system 10 to further ensure accurate placement of the system. Further, balloon catheter system 10 may be filled with contrast die for enhanced visualization prior to deployment of balloon 20. Balloon 20 is affixed to a polymer jacket 22 (shown in broken line), which overlays a hypotube (such as the hypotube 40 illustrated in FIG. 2 and described in greater detail below).

Polymer jacket 22 may be, for example, a PEBAX (polyether block amide) having a hardness of 35-55D as measured with a durometer. Jacket 22 may be applied utilizing heat shrinking and distributed over the length of the hypotube. The jacket 22 may optionally stop just short of the distal end of the underlying hypotube. Polymer jacket 22 provides a fluid tight seal over the hypotube. Further, it provides a bonding substrate to which the balloon 20 is bonded at or near the distal end of the hypotube and system 10. A hydrophilic coating may be applied over most or all of the polymer jacket 22, balloon 20, and the exterior of balloon catheter system 10. Polymer jacket 22 provides a tie layer for the hydrophilic coating, which may enhance deliverability of the system 10.

Though balloon 20 is illustrated in its inflated configuration in FIG. 1, during tracking of balloon catheter system 10 to a treatment site, balloon 20 is not fully inflated. During tracking of balloon catheter system 10 to a treatment site, the device is in its delivery configuration, and balloon 20 will be fully or partially deflated and may be completely or partially folded or crimped. In such a delivery configuration (not pictured), prior to inflation of balloon 20, the delivery profile of balloon catheter 10 is approximately equal to 0.018 inch. Accordingly, balloon catheter system 10 can be delivered to a treatment site using a guide catheter as small as 1.5 French.

An example of a hypotube component suitable for use in construction of balloon catheter system 10 is illustrated in FIG. 2. FIG. 2 illustrates a laser cut or profiled machine cut NiTi (Nitinol®) hypotube 40 which may alternatively be constructed from any number of compositions having suitable biocompatibility and strength characteristics. Alternative suitable metals include stainless steel such as, for example, 316L SS, and cobalt chrome for enhanced visibility.

An exemplary hypotube 40 has an approximate inner diameter of 0.009 inch, and an approximate outer diameter of 0.014 inch, but may be dimensioned in any number of suitable sizes and lengths depending upon the entry point into the vasculature, the location of the aneurysm, variances in patient anatomy, and any extenuating circumstances. Hypotube 40 may desirably be cut using an oxygen laser to remove oxide from either the inner or outer surface of hypotube 40. The cut pattern of hypotube 40 includes a first helical cut 46 typically having a varied pitch from proximal end 50 to distal end 52. Pitch will be understood to mean the proximity of successive cut lines, with increasing pitch referring to increasing proximity. The first helical cut 46 typically has a pitch increasing as it approaches distal end 52, to confer increased flexibility at the distal end. (The pattern appears as dotted lines where it would appear on the opposite side of hypotube 40 as though “seen through” hypotube 40.) The increasing pitch approaching the distal end 52 will be selected to confer the desired support profile on the tube. Hypotube 40 is cut with a second helical cut 48, typically being 180° out of phase from the first helical cut 46, thus avoiding cross-over of the cuts, and following the same variation in pitch from proximal end 50 to distal end 52. The offset spiral cuts 46 and 48 provide an interference locking mechanism to confer stretch resistance in hypotube 40 while at the same time preserving lateral flexibility. The offset spiral cuts 46 and 48 decrease in pitch approaching proximal end 50, until they terminate to finish on a solid tube (not pictured). Additional (third, fourth, etc.) helical cuts may be made to further enhance stretch resistance and lateral flexibility. For example, a third spiral cut out of phase by 120° may be made. After forming the desired spiral cuts, the cut hypotube 40 is ready for application of a polymer jacket, hydrophilic coating and balloon attachment.

After sterilization, a system manufactured according to the invention may be utilized in any one of a number of intravascular procedures. In a typical procedure according to the invention, a guide catheter is introduced into the femoral artery and navigated through the vascular system under fluoroscopic visualization. The guide catheter may be as small as 1.5 French. The distal end of the guide catheter is positioned near the proposed treatment site within the vasculature or other luminal structure of a subject. (The treatment site may be, for example, an aneurysm, an arterio-venous malformation, an occlusion, or other defect.) The balloon catheter system according to the invention is then advanced to the treatment site through the guide catheter. The catheter is then placed proximate the defect, and the balloon inflated.

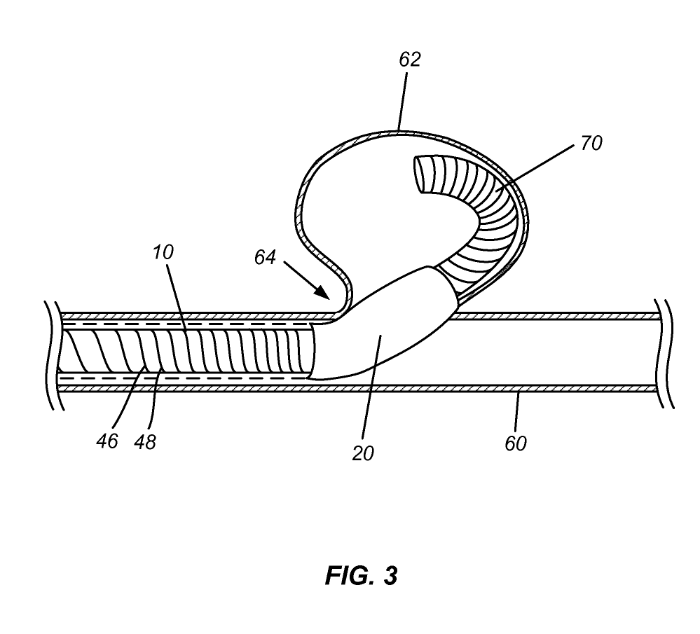

In the example illustrated in FIG. 3, an inflatable balloon catheter system 10 has been tracked to the treatment site within vessel 60 via a guide catheter (not shown). Aneurysm 62 is disposed within vessel 60. The balloon 20 disposed near the distal end of the balloon catheter system 10 may be suitably and safely positioned at the treatment site without the use of a guide wire. For example, as illustrated in FIG. 3, the balloon 20 can be positioned at the neck 64 of the aneurysm 62. The inflatable balloon 20 can then be inflated to remodel the neck 64 of the aneurysm 62 and to secure balloon 20 of catheter system 10 across the neck of aneurysm 62.

Following inflation of balloon 20, an embolic coil 70 or other suitable embolic material (not pictured) may be delivered to the aneurysm 62 through the lumen of the catheter tube 12. Balloon 20 holds system 10 in place during delivery of embolic material, and further prevents escape of embolic coils or material from aneurysm 62 into vessel 60 during delivery. Upon completion of delivery of embolic coil 70 to aneurysm 62, balloon 20 may be deflated and withdrawn from vessel 60 and from the subject.

While the invention may be modified and alternative forms may be used, specific embodiments of the invention have been illustrated and described in detail. It should be understood, however, that the description herein of specific embodiments is not intended to limit the invention to the particular forms disclosed. The invention and following claims are intended to cover all modifications and equivalents falling within the spirit and scope of the invention.

What is claimed is:

1. A catheter for use in intravascular procedures, the system comprising:
a tubular element having a proximal end and a distal end, a first helical cut and a second helical cut, wherein said first cut is out of phase from said second cut so that said cuts do not intersect;
a polymer jacket disposed about the tubular element; and
an elastomeric balloon bonded to said polymer jacket.

2. The catheter according to claim 1, wherein said first and second helical cut patterns are out of phase by an angle in the range from 90° to 180°.

3. The catheter according to claim 1, wherein said helical cuts have a first pitch at said distal end and a second pitch at said proximal end, wherein said second pitch is greater than said first pitch.

4. The catheter according to claim 3, further comprising an intermediate pitch, wherein said intermediate pitch transitions in gradually increasing pitch between said first pitch and said second pitch.

5. The catheter according to claim 1, wherein said tubular element comprises nickel titanium, stainless steel, or cobalt chrome.

6. The catheter according to claim 1, wherein said tubular element comprises an outer diameter of 0.015 inch or less.

7. The catheter according to claim 1, wherein said tubular element comprises an inner diameter of 0.010 inch or less.

8. The catheter according to claim 1, wherein said system is deliverable through a guide catheter of 1.5 French.

9. The catheter according to claim 1, wherein said elastomeric balloon has a stiffness and pliability sufficient for inflation of said balloon at an intracranial aneurysm without a guide wire.

10. The catheter according to claim 9, wherein said tubular member has a lumen and a distal port for delivery of one or more embolic implants.

11. The catheter according to claim 10, wherein said balloon comprises a proximal end and a distal end and said distal port is disposed at or beyond the distal end of said balloon.

12. The catheter according to claim 10, wherein said balloon comprises a proximal end, a distal end and an intermediate length, wherein said distal port is disposed along said intermediate length.

13. The catheter according to claim 1, wherein said balloon is inflatable using inflation media and said balloon further comprises means for slowly releasing said inflation media.

14. The catheter according to claim 1, wherein said polymer jacket is PEBAX having a durometer between 35D-55D.

15. The catheter according to claim 1, wherein said polymer jacket provides a fluid tight seal to said catheter system.

16. A method of manufacture of a catheter comprising the steps of:
providing a metal tubular member having a cylindrical wall;
cutting a first helix through the wall of the tubular member;
cutting a second out-of-phase, non-intersecting helix through the wall of the tubular member;
affixing a polymer jacket over the exterior of tubular member; and
attaching an inflatable elastomeric balloon to a distal end of the tubular member or polymer jacket.

17. The method according to claim 16, wherein said second helix is out-of-phase with first helix by between 90° and 180°.

18. The method according to claim 16, further comprising cutting a third helix in the cylindrical wall, said third helix being out-of-phase and non-intersecting with the first and second helices.

19. The method according to claim 16, wherein said step of cutting comprises cutting with a laser.

20. The method according to claim 16, with the additional step of applying a hydrophilic coating to the exterior of the polymer jacket.

21. The method according to claim 16, wherein said polymer jacket comprises a polyether block amide with a durometer between 35D-55D.

22. The method according to claim 16, wherein said polymer jacket covers the entire length of the tubular member.

23. The method according to claim 16, wherein said tubular element comprises a proximal end and a distal end and said first helix and said second helix are each cut in a helical pattern with a gradually increasing pitch from said proximal end to said distal end.

24. The method according to claim 16, wherein said elastomeric balloon comprises radiopaque material.
